(19) United States
(12) Patent Application Publication
(10) Pub. No.: US 2010/0331958 A1
(43) Pub. Date: Dec. 30, 2010

(54) HINGED ENDOVASCULAR DEVICE

(75) Inventor: Michael V. Chobotov, Santa Rosa, CA (US)

Correspondence Address: GRANT ANDERSON LLP, GRANT ANDERSON LLP C/O PORTFOLIOIP, P.O. BOX 52050, MINNEAPOLIS, MN, 55402, US

(73) Assignee: Trivascular, Inc.

(21) Appl. No.: 12/747,499

(22) PCT Filed: Dec. 19, 2008
(86) PCT No.: PCT/US08/87831

§ 371 Date: Sep. 7, 2010

Related U.S. Application Data
(60) Provisional application No. 61/015,499, filed on Dec. 20, 2007.

(51) Int. Cl. A61F 2/82

(52) U.S. Cl. 623/1.15

(57) ABSTRACT

A hinged endovascular device including a first stent portion having a first proximal end and a first distal end and a second stent portion having a second proximal end and a second distal end. A hinge assembly couples the first distal end and the second proximal end to each other. A method of inserting the device into a body lumen is also disclosed.

### RELATED APPLICATIONS

This application is a National Stage Application under 35 U.S.C. section 371 of international patent application number PCT/US2008/087831, filed Dec. 19, 2008, by Michael V. Chobotov, titled “Hinged Endovascular Device”, and designated by attorney docket number TRI-1424-PC, which also claims priority from U.S. Patent Application Ser. No. 61/015,499, filed Dec. 20, 2007, by Michael V. Chobotov, titled “Hinged Endovascular Device”, both of which are incorporated by reference herein in their entirety.

### BACKGROUND OF THE INVENTION

An endovascular device, such as a stent, may be inserted into a body lumen, such as an artery, to open the artery or to provide structural support to the artery in the area of an aneurysm. Often, the stent is introduced into a relatively small artery, such as a femoral artery, and advanced to a relatively larger artery for final placement, such as the abdominal artery. In order to adequately seal off the aneurysm, the stent must be large enough to span the larger artery after the stent is expanded. The required size of the stent may inflict excessive trauma to the smaller artery that is being used to deliver the stent to the larger artery.

There exists a need to be able to provide a stent to seal and bypass an aneurysm in a relatively large body lumen, wherein the stent has a sufficiently small profile so as not to inflict excessive trauma to the smaller body lumen that is being used as a conduit to deliver the prosthesis.

### SUMMARY OF THE INVENTION

Briefly, the present invention provides in one aspect a hinged stent comprising a first stent portion having a first proximal end and a first distal end and a second stent portion having a second proximal end and a second distal end. A hinge assembly couples the first distal end and the second proximal end to each other.

Also, the present invention provides in another aspect a method of inserting a hinged prosthesis into a body lumen having a main lumen with first and second lumens extending therefrom at a bifurcation. The method comprises the steps of inserting a prosthesis having a first portion, a second portion, and a hinged portion coupling the first portion to the second portion into the first lumen distally of the bifurcation; advancing the first portion of the prosthesis toward the bifurcation; pivoting the first portion of the prosthesis into the second lumen at the bifurcation; advancing the first portion into the second lumen while advancing the hinged portion toward the bifurcation; and advancing the hinged portion in a proximal direction away from the bifurcation.

The present invention further provides in yet another aspect a hinged stent graft assembly comprising a first stent portion having a first proximal end, a first distal end, and a first graft portion at least partially covering the first stent portion. A second stent portion has a second proximal end, a second distal end, and a second graft portion at least partially covering the second stent portion. A hinge couples the first graft portion and the second graft portion to each other.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain features of the invention. In the drawings:

FIG. 1 is a front elevational view of a stent assembly according to an exemplary embodiment of the present invention;

FIG. 2 is an end profile view of the stent assembly of FIG. 1, taken along lines 2-2 of FIG. 1;

FIG. 3 is a front elevational view, partially in section, of the stent assembly of FIG. 1 inserted into a body lumen;

FIG. 4 is a perspective view of an alternative embodiment of a stent assembly according to the present invention;

FIG. 5 is a side elevational view, partially in section, of the stent assembly of FIG. 1 loaded into a delivery device;

FIG. 6 is a front elevational view, partially in section, showing a guidewire having been inserted into the body lumen of FIG. 3;

FIG. 7 is an enlarged front elevational view, partially in section, of the delivery device of FIG. 5 being inserted over the guidewire of FIG. 6;

FIG. 8 is an enlarged front elevational view, partially in section, of the delivery device of FIG. 5 being removed from the stent assembly of FIG. 1;

FIG. 9 is an enlarged front elevational view, partially in section, of the stent assembly of FIG. 1 being urged into a desired deployment location; and

FIG. 10 is a front elevational view of a stent assembly according to an alternative exemplary embodiment of the present invention.

### DETAILED DESCRIPTION OF THE INVENTION

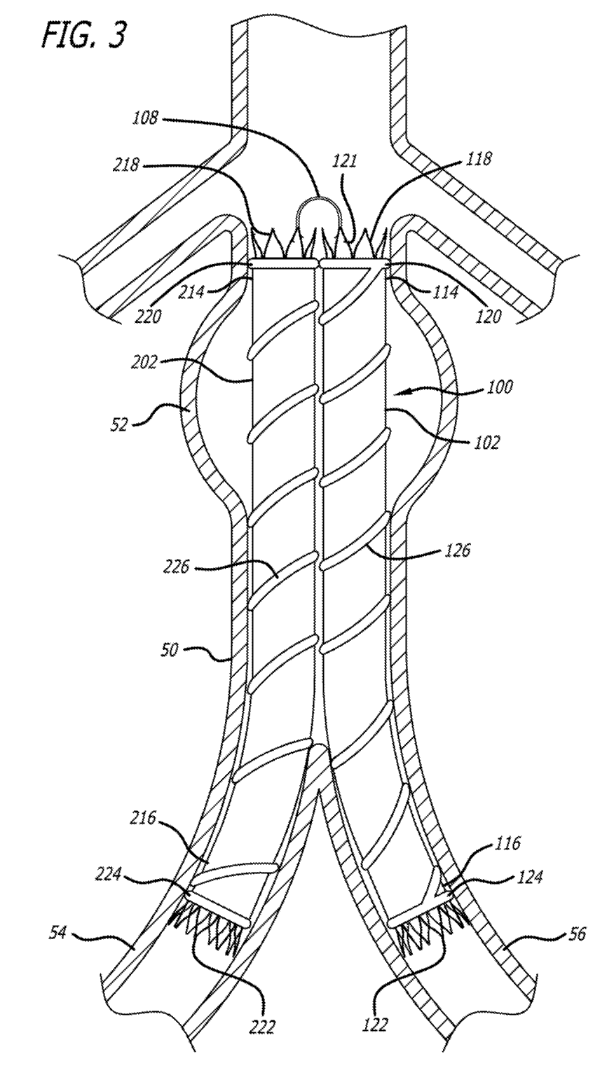

Certain terminology is used in the following description for convenience only and is not limiting. As used herein, the term “distal” is defined to mean the direction toward the bottom of FIG. 3 and the term “proximal” is defined to mean the direction toward the top of FIG. 3. The terminology includes the words above specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Referring to the figures in general, a hinged endovascular prosthesis 100 according to exemplary embodiments of the present invention are shown. Prosthesis 100 may be a stent, a graft, a stent-graft, or other endoluminal prosthesis, including but not limited to a vena cava filter, or other such device. In exemplary embodiments described in detail below, prosthesis 100 may be a stent-graft that may be used to bypass an aneurysm in a body lumen, such as an abdominal aortic aneurysm. Those skilled in the art, however, will recognize that prosthesis 100 may be used to treat other body lumens, such as bifurcated coronary arteries, and other maladies, as well.

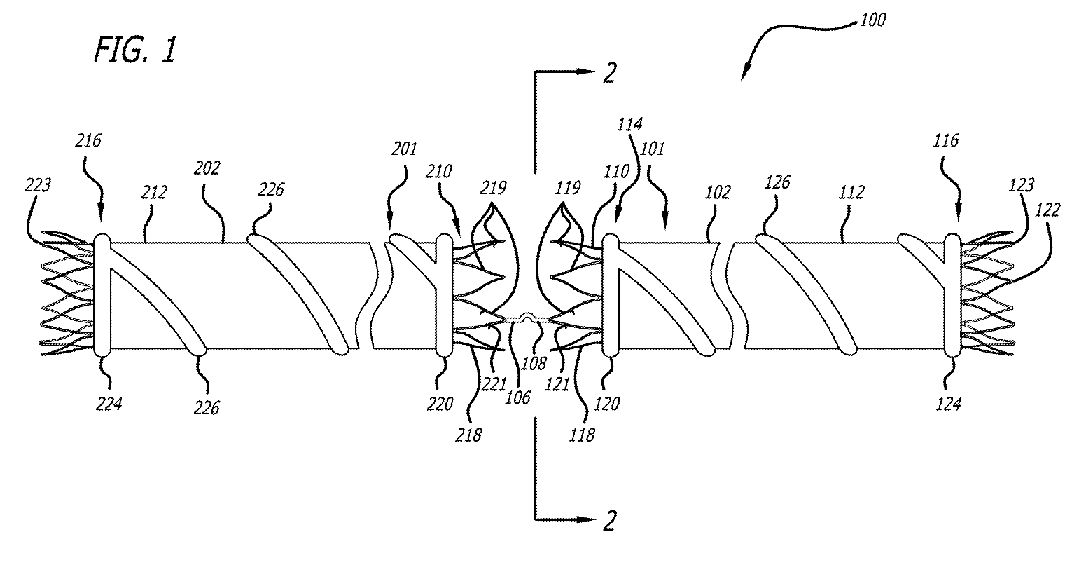

Referring to FIG. 1, prosthesis 100 includes a contralateral, or first, stent portion 102, an ipsalateral, or second, stent portion 202, and a hinge assembly 106 that hingedly couples first stent portion 102 to second stent portion 202. Hinge assembly 106 allows first stent portion 102 to rotate approximately 180 degrees relative to second stent portion 202.

Elements of first stent portion 102 are indicated with numerals 1xx, while like elements of second stent portion 202 are indicated with numerals 2xx. A description of an element of first stent portion 102 also describes a corresponding element of second stent portion 202.

First stent portion 102 includes a stent 110 that is at least partially surrounded by a graft 112. Graft 112 may be constructed from various materials, for example, expanded PTFE and may be formed by blow-forming/sintering, or other known methods. Graft 112 may be coupled to stent 110 by known methods.

Stent 110 may have a tapered body such that a proximal end 114 of stent 110 is larger than a distal end 116 of stent 110. Stent 110 may be self expanding, balloon expanding, or a combination of self expanding and balloon expanding. Stent 110 may be constructed from a self expanding material such as, for example, nitinol, or other suitable known self expanding material. In an exemplary embodiment, stent 110 may be cut from a sheet. Other known manufacturing processes, such as welding, blow-forming/sintering of grafts 112, 212 may be used. Further, the invention disclosed herein may also be applied to non-inflatable stent-grafts.

Proximal end 114 of stent 110 includes a proximal crown 118 that extends proximally of graft 112. Proximal crown 118 may include 4 individual crown elements 119, although those skilled in the art will recognize that more or less than 4 individual crown elements 119 may be used. Crown elements 119 may also include barbs 121 to prevent prosthesis 100 from migrating after placement within a lumen. Proximal end 114 of stent 110 also includes a proximal cuff 120 that may be inflated after insertion of prosthesis 100 in a desired location.

Distal end 116 of stent 110 includes a distal crown 122. Distal crown 122 may include 8 individual crown elements 123, although those skilled in the art will recognize that distal crown 122 may use more or less than 8 individual crown elements 123. Distal end 116 also includes a distal cuff 124. An inflation channel 126 provides fluid communication between distal cuff 124 and proximal cuff 120.

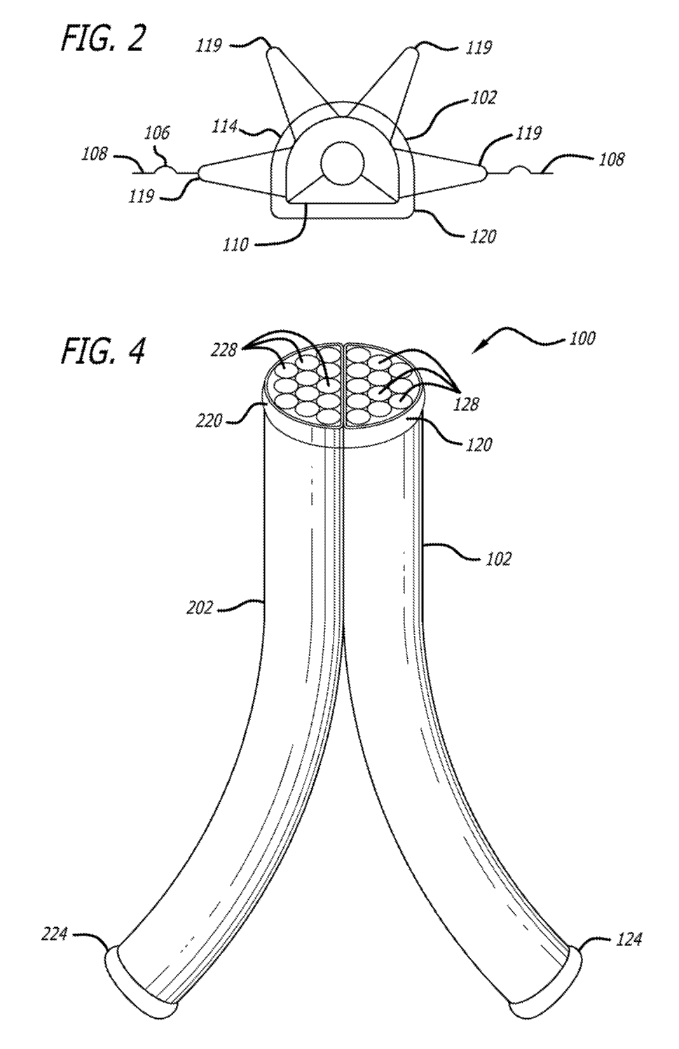

As shown in FIG. 2, a view of proximal end 114 of first stent portion 102 shows proximal crown 118 with 4 individual crown elements 119. Also, proximal end 114 of stent 110 has a generally “D-shaped” configuration, with proximal crown 118 surrounding the curved portion of the D-shape. The flat portion of the D-shaped is free of crown elements 119.

Hinge assembly 106 includes hinges 108 that extend from diametrically opposed proximal crown elements 119 and couple to corresponding crown elements 219 (shown in FIG. 1). Hinges 108 may be generally “dogleg” shaped to facilitate pivoting of first stent portion 102 relative to second stent portion 202 during insertion into a body lumen. Alternatively, although not shown, crown elements 119, 219 may be enlarged at the diametrically opposed locations and coupled directly to each other to serve as hinges.

FIG. 3 shows prosthesis 100 having been inserted into a bifurcated body lumen, such as aortic lumen 50, having an aneurysm 52. Aortic lumen 50 splits into branched ipsalateral lumen 54 and branched contralateral lumen 56. Prosthesis 100 spans aneurysm 52, forming a passageway through aneurysm 52 between aortic lumen 50 and each of the ipsalateral lumen 54 and contralateral lumen 56.

Prosthesis 100 is located within aortic lumen 50 such that the flat portions of the “D-Shaped” of the proximal end 114, 214, respectively, of each of the first stent portion 102 and second stent portion 202 are juxtaposed against each other. Proximal cuffs 120, 220 on each of first stent 102 and second stent 202 are inflated to seal proximal ends 114, 214 of prosthesis 100 against aortic lumen 50 to preclude blood flow between prosthesis 100 and aortic lumen 50.

Distal end 116 of first stent portion 102 extends into ipsalateral lumen 54. Distal end 116 of stent 110 may have a generally circular cross section to facilitate sealing distal end 116 against contralateral lumen 56. Distal cuff 124 is inflatable to seal distal end 116 of first stent portion 102 against the walls of contralateral lumen 56, precluding fluid flow between first stent portion 102 and contralateral lumen 56. Similarly, distal end 216 of second stent portion 202 extends into ipsalateral lumen 54. Distal cuff 224 of second stent portion 202 is inflatable to seal against the walls of ipsalateral lumen 54, precluding fluid flow between second stent portion 202 and ipsalateral lumen 54. In this embodiment, cuffs 120, 124 do not fluidly communicate with cuffs 220, 224.

After insertion of prosthesis 100 as shown in FIG. 3, single lumen branch stent grafts (not shown) may be coupled to distal ends 116, 216 of each of first stent portion 102 and second stent portion 202, respectively, according to known methods.

The separation of prosthesis 100 into first stent portion 102 and second stent portion 202 reduces the cross sectional profile of prosthesis 100 and, consequentially its delivery system, during insertion through ipsalateral lumen 54 into aortic lumen 50. Such reduction in the cross sectional size of prosthesis 100 may result in reduced trauma to the patient during insertion of prosthesis 100.

In an alternative embodiment, shown in FIG. 4, a plurality of straws 128, 228 may be inserted into the interior lumen of each of first stent portion 102 and second stent portion 202. Straws 128, 228 may provide additional support to prosthesis 100 in each of first stent portion 102 and second stent portion 202.

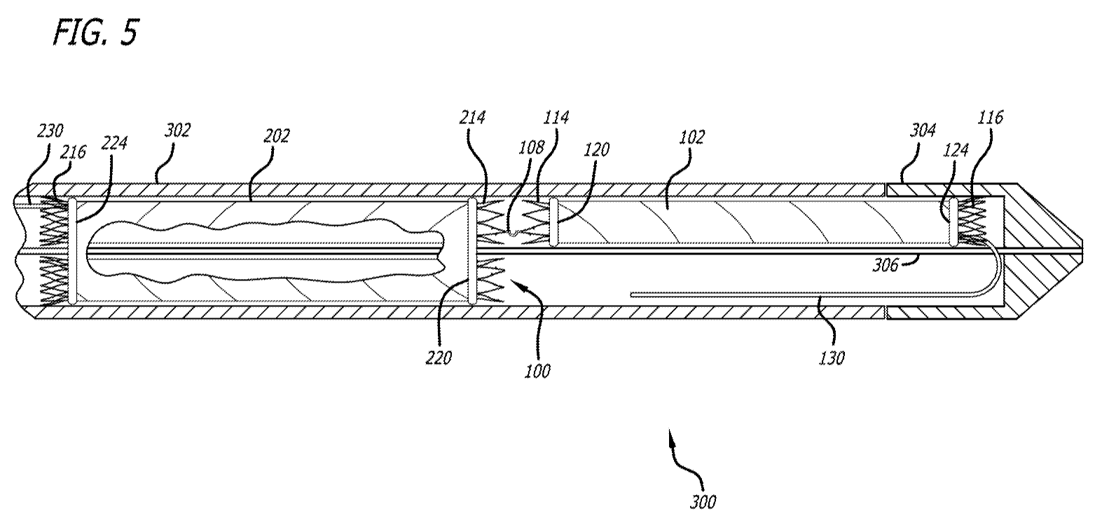

Insertion of prosthesis 100 into lumen 50 is described below and shown in FIGS. 5-9. FIG. 5 shows prosthesis 100 loaded into a delivery sheath assembly 300. Sheath assembly 300 includes a delivery sheath 302 and a nosecone 304. Nosecone 304 includes a guidewire lumen 306 that extends through sheath 302. Prosthesis 100 is inserted into sheath assembly 300 such that guidewire lumen 306 does not extend through first stent portion 102, but extends through second stent portion 202. Guidewire lumen 306 does not pass through first stent portion 102 so that, after unsheathing first stent portion 102 from sheath 302, first stent portion 102 can bend away from guidewire lumen 306. Optionally, guidewire lumen 306 may be hinged (not shown) to facilitate movement of guidewire lumen 306 between ipsalateral lumen 54 and contralateral lumen 56.

Prosthesis 100 is inserted into sheath assembly 300 such that distal end 116 of first stent portion 102 is proximate to nosecone 304 and proximal end 114 of first stent portion 102 is proximate to proximal end 214 of second stent portion 202. While first stent portion 102 is shown in FIG. 5 to be compressed to lay on one side of guidewire lumen 306, those skilled in the art will recognize that first stent portion 102 may wrap around guidewire lumen 306 without guidewire lumen 306 extending through first stent portion 102. A contralateral leash 130, which is used to inflate cuffs 120, 124, extends from distal cuff 124 and is slidingly coupled to nosecone 304. An ipsalateral leash 230, which is used to separately inflate cuffs 220, 224, extends from distal cuff 224.

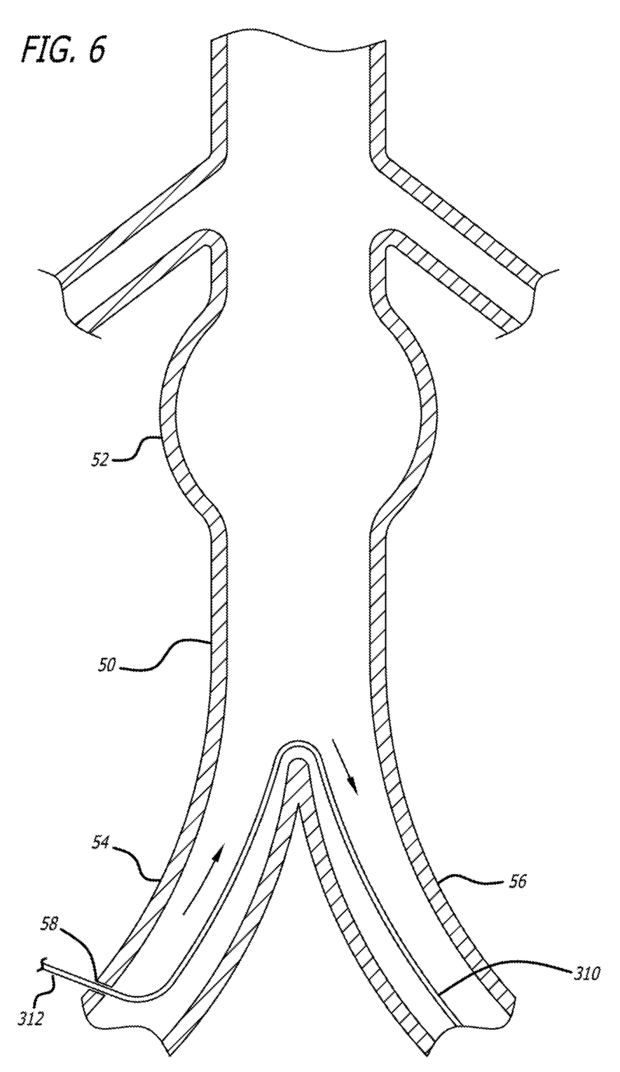

To insert prosthesis 100, as shown in FIG. 6, a guidewire is 310 inserted into ipsalateral lumen 54 through an incision 58 in the wall of ipsalateral lumen 54 and fed up to aortic lumen 50. Guidewire 310 is then fed distally through contralateral lumen 56.

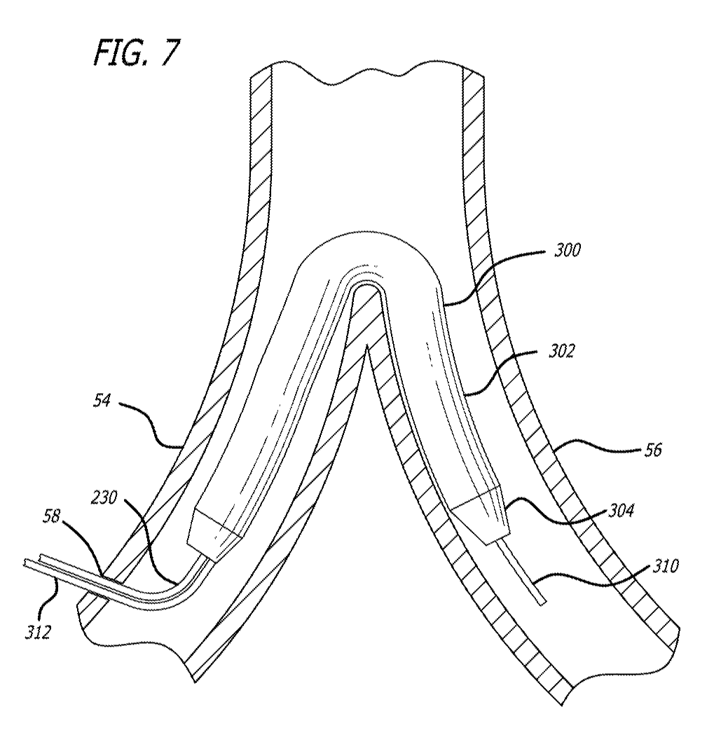

Next, as shown in FIG. 7, sheath assembly 300 is fed into ipsalateral lumen 54 over distal end 312 of guidewire 310 and is then advanced into contralateral lumen 56. Ipsalateral leash 230 is shown extending through incision 58.

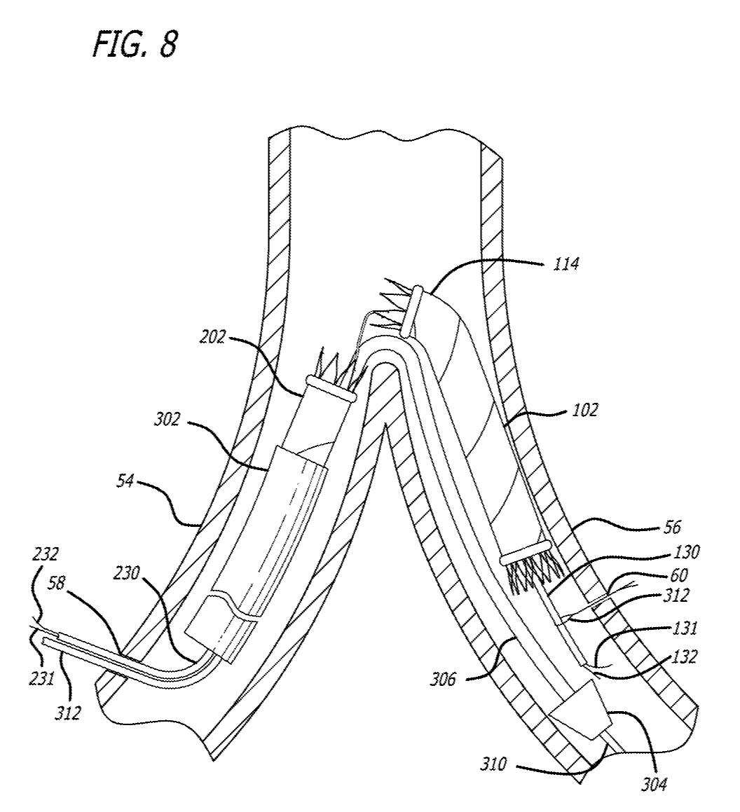

FIG. 8 shows nosecone 304 (with guidewire lumen 306) having been advanced distally into contralateral lumen 56. As nosecone 304 advances distally into contralateral lumen 56, contralateral leash 130, which contains a contralateral fill tube 131 and a release wire 132, advances distally into contralateral lumen 56 with nosecone 304. Contralateral leash 130 may be snared by a snare 312 inserted into contralateral lumen 56 through an incision 60. FIG. 8 also shows sheath 302 having been retracted distally through contralateral lumen 56 and ipsalateral lumen 54, releasing first stent portion 102 in contralateral lumen 56 and beginning to release second stent portion 102 in ipsalateral lumen 54. After sheath 302 fully releases second stent portion 202, guidewire lumen 306 and nosecone 304 may then be retracted through contralateral lumen 56 and ipsalateral lumen 54, and back into sheath 302, so that sheath assembly 300 may be removed from the patient. Guidewire 310 may be removed from lumens 54, 56 by pulling guidewire 310 distally through incision 58.

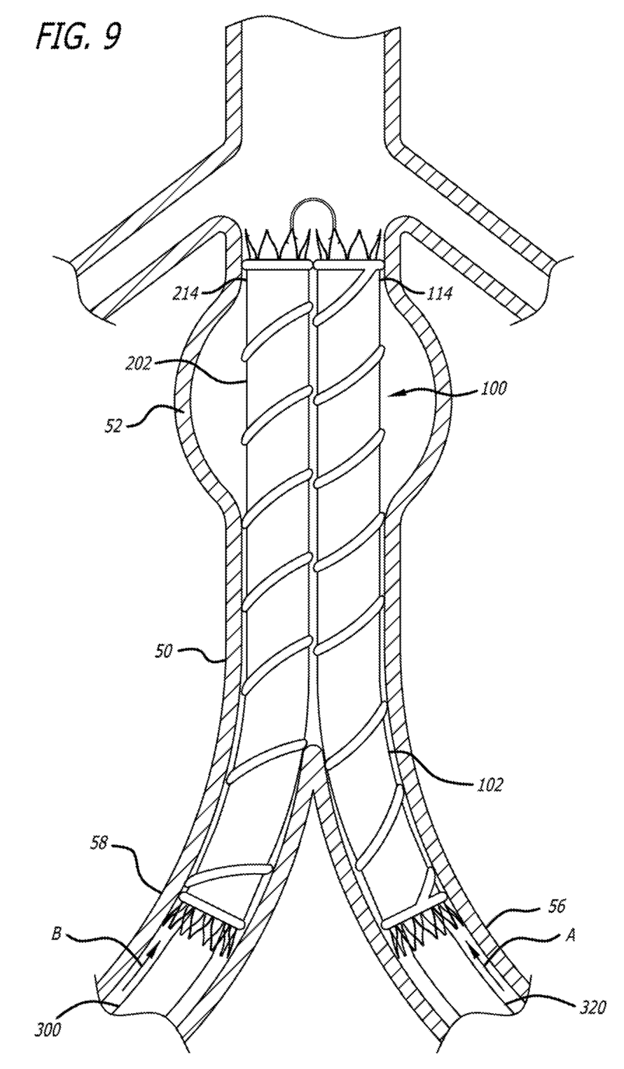

Snare 312 pulls contralateral leash 130 through incision 60, and an obdurator may be slid over contralateral leash 130 and advanced to first stent portion 102. As shown in FIG. 9, obdurator 320 may be used to urge proximal end 114 of first stent portion 102 past aneurysm 52 in the direction of arrow “A” to its desired location. Simultaneously with placement of first stent portion 102, nosecone 304 may be advanced proximally in the direction of arrow “B” to urge proximal end 214 of second stent portion 202 past aneurysm 52 to its desired location. Hinges 106 assist in moving proximal ends 114, 214 of each of first stent portion 102 and second stent portion 202 together.

After prosthesis 100 has been moved to a desired location, such as is shown in FIG. 9, cuffs 120, 124 and 220, 224 may be inflated via fill tubes 131, 231, respectively, using the inflation mechanisms disclosed in U.S. Pat. No. 6,761,733, which is owned by the assignee of the present invention, and which is incorporated herein by reference in its entirety. In an exemplary embodiment, distal end of contralateral fill tube 131 may be coupled to a syringe (not shown) containing an expansion fluid, such as, for example, saline. Expansion fluid is forced through contralateral fill tube 131 and into distal cuff 124, expanding distal cuff 124, connection tube 126, and proximal cuff 120. After cuffs 124, 120 are filled, release wire 132 may be pulled distally, releasing fill tube 131 from cuff 124, allowing release wire 132 and fill tube 131 to be distally removed from ipsalateral lumen 54 through incision 60.

Distal end of ipsalateral fill tube 231 may be coupled to a syringe (not shown) containing an expansion fluid. Expansion fluid is forced through ipsalateral fill tube 231 and into distal cuff 224, expanding distal cuff 224, connection tube 226, and proximal cuff 220. After cuffs 224, 220 are filled, release wire 232 may be pulled distally, releasing fill tube from cuff 224, allowing release wire 232 and fill tube 231 to be distally removed from ipsalateral lumen 54 through incision 58.

In an alternative embodiment, not shown, proximal cuff 220 may be in fluid communication with proximal cuff 120 via a coupling tube, that allows inflation of proximal cuff 120, connection tube 126, and distal cuff 124 via ipsalateral fill tube 231, eliminating the need for contralateral fill tube 131 and its associated release wire 132.

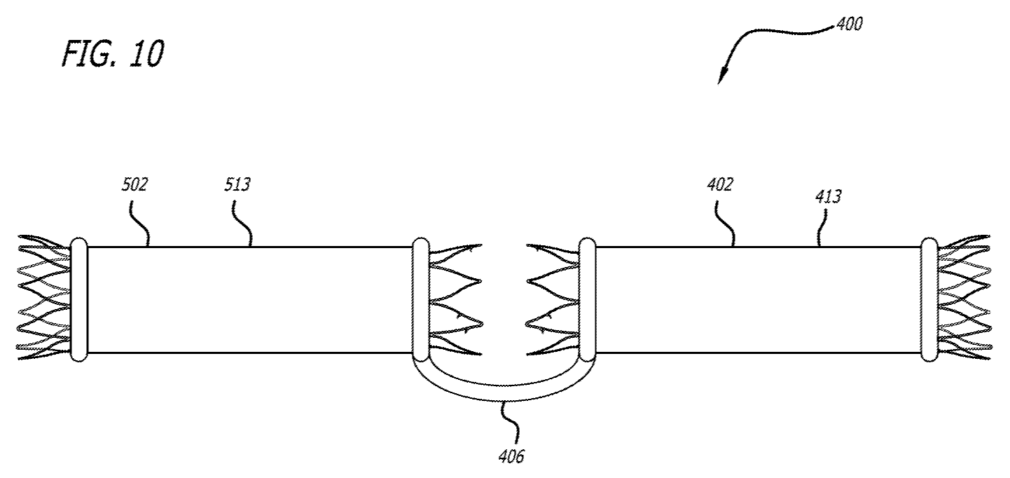

As shown an alternative embodiment of a prosthesis 400 according to the present invention in FIG. 10, a first stent portion 402 and a second stent portion 502 need not be coupled together by hinge assembly 106. Prosthesis 400 has a first graft 413 that at least partially covers first stent portion 402 and a second graft 513 that at least partially covers second stent portion 502. A hinge assembly 406 constructed from graft material couples graft 413 to graft 513. Prosthesis 400 is inserted into the patient in the same manner as prosthesis 100 described above.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A hinged stent comprising:
a first stent portion having a first proximal end and a first distal end;
a second stent portion having a second proximal end and a second distal end; and
a hinge assembly coupling the first distal end and the second proximal end to each other.

2. The hinged stent according to claim 1, further comprising a first graft coupled to the first stent portion.

3. The hinged stent according to claim 1, further comprising a second graft coupled to the second stent portion.

4. The hinged stent according to claim 1, wherein the hinge assembly comprises first and second hinges diametrically opposed from each other.

5. A method of inserting a hinged prosthesis into a body lumen having a main lumen with first and second lumens extending therefrom at a bifurcation, the method comprising the steps of:
(a) inserting a prosthesis having a first portion, a second portion, and a hinged portion coupling the first portion to the second portion into the first lumen distally of the bifurcation;
(b) advancing the first portion of the prosthesis toward the bifurcation;
(c) pivoting the first portion of the prosthesis into the second lumen at the bifurcation;
(d) advancing the first portion into the second lumen while advancing the hinged portion toward the bifurcation; and
(e) advancing the hinged portion in a proximal direction away from the bifurcation.

6. The method according to claim 5, wherein step (e) comprises advancing the hinged portion to a location in the main lumen proximal of the aneurysm.

7. A hinged stent graft assembly comprising:
a first stent portion having a first proximal end and a first distal end;
a first graft portion at least partially covering the first stent portion;
a second stent portion having a second proximal end and a second distal end;
a second graft portion at least partially covering the second stent portion; and
a hinge coupling the first graft portion and the second graft portion to each other.
